(19) United States
(12) Patent Application Publication
(10) Pub. No.: US 2011/0200498 A1
(43) Pub. Date: Aug. 18, 2011

(54) SYSTEM AND METHOD FOR PACKAGING DRY TEST STRIPS

(75) Inventor: Robert Huffstodt, Indianapolis, IN (US)

(73) Assignee: Polymer Technology Systems, Inc., Indianapolis, IN (US)

(21) Appl. No.: 12/999,905

(22) PCT Filed: Jun. 17, 2009
(86) PCT No.: PCT/US09/47653

§ 371 Date: Apr. 25, 2011

Related U.S. Application Data
(60) Provisional application No. 61/073,242, filed on Jun. 17, 2008.

(51) Int. Cl. G01N 33/48; B65B 61/20

(52) U.S. Cl. 422/401; 53/445

(57) ABSTRACT

A plurality of different types of dry strips are packaged in a single-user container. Strip-specific scheduling information for using the dry test strips is placed either in or on the container. The scheduling information may be provided to the packager by a physician and may be stored on an electronically or optically readable storage device packaged with the test strips. The storage device may be a memory, which memory may be inserted into a dry test strip reader and the scheduling information may be communicated to the user either visually or audibly.

### BACKGROUND OF THE INVENTION

1. Field of the Invention

The invention in general relates to disposable dry test strips for testing bodily fluids, and more particularly to a method and apparatus for packaging such test strips.

2. Statement of the Problem

Dry test strips are well known in the art. For example, dry test strips to determine glucose levels in the blood may be used several times a day by diabetics. Lipid and lipid-related dry test strips are used to determine the concentration of a particular blood lipid or lipid related analyte, such as total cholesterol, high density lipoprotein cholesterol (HDL), low density lipoprotein cholesterol (LDL), triglycerides, and ketones. See, for example, U.S. Pat. No. 5,597,532 issued Jan. 28, 1997 to James Connolly, U.S. Pat. No. 5,366,609 issued to Bradley E. White et al. on Nov. 22, 1994, U.S. Pat. No. 5,246,858 issued to Steven R. Arbuckle et al. on Sep. 21, 1993, U.S. Pat. No. 5,104,619 issued to Aurora F. de Castro et al. on Apr. 14, 1992, and U.S. Pat. No. 5,053,199 issued to Dale A Keiser et al. on Oct. 1, 1991. It is noted that ketones are not lipids, but are related to lipids in that they are a by-product of the burning of lipids by the body. One form of state-of-the-art dry test strips, such as lipid and lipid related dry test strips, generally include a dispersion layer to which the blood is applied, one or more red blood cell layers beneath the dispersion layer for separating the red blood cells from the blood plasma, a reagent layer beneath the red blood cell layer in which the reagents that isolate the particular blood analyte are dissolved by the plasma, and a bottom detection layer that produces the signal indicative of the concentration of the analyte. This signal may be, for example, a color, the density of which is proportional to the concentration of the analyte. The concentration signal is read by a test strip reader to produce an output indicating the concentration of the analyte. For example, the reader may be a reflectance meter which reads the color generated in the detection layer. Other dry test strips provide an output via a resistance measurement or other electrochemical measurement. In state-of-the-art dry test systems, a ROM chip is included with each container of dry test strips, and the strip reader includes a port into which the ROM may be inserted. See the patents cited above. The ROM contains calibration data relating to the particular batch of strips, which data is read by the reader to calibrate the output. Dry test strip systems have evolved in the last ten years to become nearly as accurate as laboratory tests run by skilled laboratory technicians.

Dry test strips have the advantage that relatively unskilled people at the site where the test is ordered or needed can perform them in a few minutes. Yet, most of the use of lipid and lipid-related dry test strips is presently limited to physician's office and hospitals. This is due to several reasons, one being the fact that use strategies for lipid and lipid-related dry test strips is somewhat more complicated then, for example, glucose test strips. For example, HDL cholesterol, LDL cholesterol, and triglyceride levels are used by physicians as a significant indicator of risk of coronary heart disease. No one of these by itself is sufficient to tell the physician what the physician needs to know for diagnosis. Ketones may be an indicator of the onset of diabetes, but can also be a normal result of dieting, hard physical exercise, and other common practices. Thus, one test is often not sufficient for a reliable diagnosis. Another reason is that, as compared to, for example, glucose, lipid and lipid-related levels in the blood change relatively slowly. Thus, physicians tend to schedule these tests to be taken at their office, where they have more control over timing and other factors. Moreover, since distributing and selling strips one or two at a time would add significantly to the distribution and sales costs, thereby significantly adding to the cost of the product, manufacturers package the test strips, like other common health or medical goods, in containers of a dozen or more which are conventionally sold to physicians.

For the above reasons, it would be desirable if a way could be found to take advantage of the accuracy of state-of-the-art dry test strips and the fact that they are easily used by unskilled end users, while still taking account of the more complicated aspects of the diagnoses based on them. If at the same time this could be done without significant increase in cost, this would be even more desirable.

### SUMMARY OF THE INVENTION

The present invention overcomes the above and other problems by packaging multiple types of dry test strips in a single-user package. The present invention also overcomes the above and other problems by packaging the strips in combinations found to be useful for diagnostic purposes. For example, a package may contain two multiple-analyte test strips that test for the concentration of LDL, HDL, and triglycerides simultaneously, four HDL cholesterol test strips, and four LDL cholesterol test strips. Or the package may contain glucose, HDL and triglycerides test strips. Preferably, the particular combinations are as prescribed by a physician for a particular patient, or as desired by a particular end user.

Packaging the test strips with multiple types of strips in a container reduces the cost per strip of distributing and selling. In addition, since generally an individual end user does not have use for more than a few such tests over a several month or longer period, in many contexts, a multiple strip package as described in the Background of the Invention above may be a six month's or more supply for a single patient or end user. Thus, the conventional packaging is wasteful for a typical end user. More importantly, the conventional packaging system and method creates a potential problem that, over such a long period, the end user may forget to use the test strips in a timely manner so as to make diagnosis less reliable. The invention overcomes and addresses this problem by packaging the strips in a manner that automatically reminds the patient or other end user of the appropriate test schedule. Preferably, this is a schedule following the direction or prescription of a physician. For example, each test is packaged in a separate sub-package from the other included tests, and strip-specific scheduling information is applied to the individual sub-package. Preferably, the packaging is structured so that only one dry test strip at a time may be removed from the multiple-strip container.

The invention also anticipates and addresses the potential problem of a user not being able to timely follow a relatively complex use schedule by using the strip reader to remind the user of the test schedule. This problem is also addressed by the strip reader checking an inserted dry test strip against the scheduled tests, and informing the user of whether the test strip has been inserted according to the schedule. The schedule information preferably is communicated to the reader via a ROM chip packaged with the test strips, via the Internet or other wired or wireless communications system, packaged with the test strips via bar-coded test strips, or via tagging a strip with an RFID (radio frequency identification device). For example, the reader may be connected to a computer with a USB connector and the schedule downloaded via the Internet. This downloading may be automatically performed via communication data packaged with the test strip, such as on a ROM, a bar code, or an RFID device.

The invention provides a system for measuring blood analytes, the system comprising: a plurality of dry strips, the test strips including a test strip of a first test strip type for measuring a first analyte type and a second test strip of a second test strip type for measuring a second analyte type different from the first analyte type, the types of test strips selected from the group consisting of: test strips for measuring the concentration of high density lipoprotein (HDL) cholesterol, test strips for measuring the concentration of low density lipoprotein (LDL) cholesterol, test strips for measuring the concentration of lipoprotein cholesterol other than HDL and LDL, test strips for measuring total blood cholesterol, test strips for measuring a triglyceride, test strips for measuring glucose, and test strips for measuring a ketone; and a single-user container of a size that can be held in one hand by a human being, the single-user container containing the first and second test strips. Preferably, the single-user container is selected from the group consisting of a bottle, a jar, a vial, a tray, or a blister pack. Preferably, the system further comprises strip-specific scheduling information for using the plurality of different types of dry test strips, the strip-specific scheduling information either in or on the container. Preferably, the system further comprises schedule confirmation information which permits a user to confirm after a strip is removed from the container what strips have been used and the time each strip was scheduled to be used. Preferably, the strip-specific scheduling information is applied to the container. Preferably, the strip-specific scheduling information is in the container. Preferably, the system further includes an optically or electronically readable data storage element, wherein the strip-specific scheduling information is stored in the data storage element. Preferably, the data storage device is selected from the group consisting of an electronic read-only memory (ROM), a bar code, and a radio frequency identification device (RFID). Preferably, the plurality of dry test strips consists of twenty-five or less of the dry test strips in the single-user container. Preferably, one of the plurality of dry test strips consists of a three analyte test strip. Preferably, the plurality of dry test strips comprises a test strip for measuring glucose and a test strip for measuring a ketone. Preferably, the plurality of dry test strips comprises a test strip for measuring HDL cholesterol, a test strip for measuring glucose, and a test strip for measuring a triglyceride. Preferably, the plurality of dry test strips comprises an HDL cholesterol test strip, an LDL cholesterol test strip, and a total cholesterol test strip.

The invention also provides a method of packaging a dry test strips, the method comprising: providing a first type of dry test strip selected from the group consisting of: test strips for measuring the concentration of high density lipoprotein (HDL) cholesterol, test strips for measuring the concentration of low density lipoprotein (LDL) cholesterol, test strips for measuring the concentration of lipoprotein cholesterol other than HDL and LDL, test strips for measuring total blood cholesterol, and test strips for measuring a ketone; providing a second type of dry test strip selected from the group consisting of: test strips for measuring the concentration of high density lipoprotein (HDL) cholesterol, test strips for measuring the concentration of low density lipoprotein (LDL) cholesterol, test strips for measuring the concentration of lipoprotein cholesterol other than HDL and LDL, test strips for measuring total blood cholesterol, and test strips for measuring a ketone, the second type of dry test strip being a different type than the second type of dry test strip; and packaging the first type of test strip together with the second type of test strip in a single-user container of a size that can be held in one hand by a human being. Preferably, the packaging comprises sealing each of the test strips in an airtight packet and then packing the packets in the container. Preferably, the method further comprises labeling each of the packets with strip-specific scheduling information. Preferably, the packaging comprises placing each of the dry test strips in a separate compartment in the container and sealing the container. Preferably, the method further comprises labeling each of the compartments with strip-specific scheduling information. Preferably, the method further comprises passing the scheduling information from a physician to a dry test strip packager, and the placing is performed by the packager. Preferably, the method further comprises storing strip-specific scheduling information in a form accessible by a dry test strip reader, inserting one of the dry test strips in the strip reader, checking the inserted dry test strip against the strip-specific scheduling information, and informing the user of the reader of whether the test strip has been inserted according to the scheduling information.

In another aspect, the invention provides a method of providing strip-specific scheduling information to a user of a dry test strip, the method comprising: storing the strip specific scheduling information in an electronic memory; placing the electronic memory in a container with one or more dry test strips; removing the electronic memory from the container; inserting the memory into a dry test strip reader; inserting a dry test strip into the reader, and communicating the scheduling information to the user via the dry test strip reader. Preferably, the communicating is selected from the group consisting of audio communication and visual communication.

In a further aspect, the invention provides a system for measuring blood analytes, the system comprising; a dry test strip for measuring the concentration of: high density lipoprotein (HDL) cholesterol; a dry test strip for measuring a triglyceride; a dry test strip for measuring glucose; and a single-user container containing the dry test strips.

In yet another aspect, the invention provides a statin diagnostic pack comprising a single-user container holding two or more different dry test strips selected from the group consisting of an HDL cholesterol test strip, an LDL cholesterol test strip, and a total cholesterol test strip.

In an additional aspect, the invention also provides a method of scheduling a dry test strip test, said method comprising: storing strip-specific scheduling information in a form accessible by a dry test strip reader, inserting a dry test strip in said strip reader, checking said inserted dry test strip against said strip-specific scheduling information, and informing the user of said reader of whether said test strip has been inserted according to said scheduling information.

The invention not only provides a packaging system that takes advantage of the fact that dry test strips can be easily used by unskilled persons, but also permits the unskilled user to follow complex testing schedules accurately, and does this in a cost effective manner. These and other objects and benefits of the invention will become apparent from the following written description when read in conjunction with the accompanying drawing.

### BRIEF DESCRIPTION OF THE DRAWINGS

FIG. 1 illustrates five test strips in an exemplary packaging system according to the invention, prior to application of the cover seal sheet and with one dry test strip removed from the tray to illustrate scheduling information on the inside of the tray;

FIG. 2 illustrates the packaging system of FIG. 1 with the cover seal sheet applied;

FIG. 3 illustrates another exemplary dry test strip packaging system according to the invention;

FIG. 4 illustrates a further exemplary dry test strip packaging system according to the invention;

FIG. 5 illustrates another exemplary dry test strip packaging system according to the invention;

FIG. 6 is a further exemplary embodiment of the dry test strip packaging system according to the invention;

FIG. 7 illustrates a dry test strip reader and removable memory unit according to the invention; and

FIG. 8 is a flow chart illustrating an exemplary process for prescribing, packaging and using dry test strips according to the invention.

### DETAILED DESCRIPTION OF THE PREFERRED EMBODIMENT

For the purposes of promoting an understanding of the principles of the invention, reference will now be made to the embodiments illustrated in the drawings and described in the following. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains. It should also be understood that, in accordance with the patent law, the drawings are not intended to be precise engineering drawings of the invention, but rather are only intended to illustrate the invention. For example, the scale of the drawings and relative size of the various parts are generally altered so as to better illustrate the invention within the constraints of a written document such as this.

In this disclosure the term “different type of test strip” means that a strip measures a different analyte. In this context, test strips for measuring high density lipoprotein (HDL) cholesterol, test strips for measuring low density lipoprotein (LDL) cholesterol, test strips for measuring lipoprotein cholesterol other than HDL and LDL, test strips for measuring total blood cholesterol, test strips for measuring glucose, test strips for measuring triglycerides, and test strips for measuring a ketone are all “different types” of test strips because they measure different analytes.

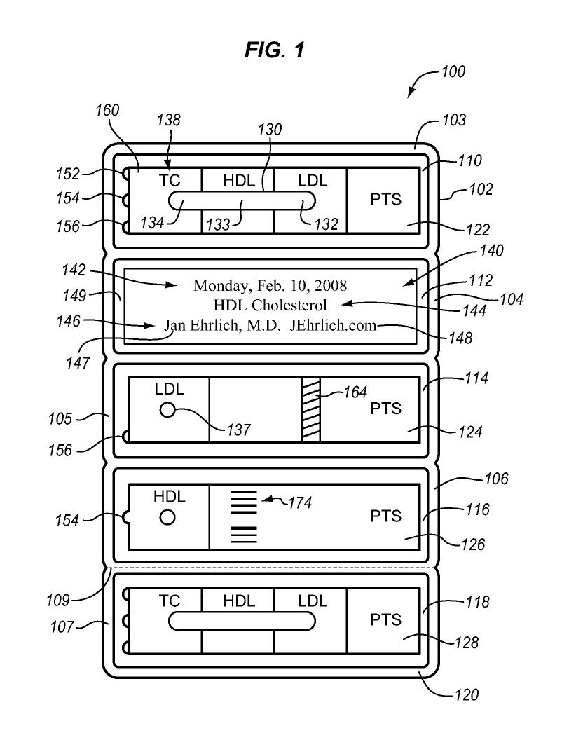

FIG. 1 illustrates a partially completed packaging system 100 according to the invention. System 100 includes a container 102, which, in this embodiment, is a packing tray 102 having five sections, 103, 104, 105, 106, and 107, each of which is separated from the other by a tear line, such as 109. Each section has a well 110, 112, 114, 116, 118 that is slightly larger than the external dimensions of a dry test strip, such as 122, that is to be packaged in the tray. The tray 102 preferably has a flat rim 120 around its periphery. In FIG. 1, well 110 holds a dry test strip of the three analyte type, that is a dry test strip 122 for measuring three different analytes, total cholesterol, HDL cholesterol, and LDL cholesterol. Well 112 is empty. Well 114 holds a dry test strip 124 of the LDL analyte type, that is, a dry test strip for measuring the concentration of LDL cholesterol. Well 116 holds a dry test strip 126 of the HDL analyte type, that is a test strip for measuring the concentration of HDL cholesterol, and well 118 holds a four analyte type dry test strip 128. The three analyte dry test strips 122 and 128 may be as described in U.S. patent application Ser. No. 10/334,043 filed Dec. 30, 2002, the LDL dry test strip may be as described in U.S. patent application Ser. No. 10/962,272 filed Oct. 11, 2004, and the HDL dry test strip may be as described in U.S. patent application Ser. No. 11/206,893 filed Aug. 17, 2005. As known in the art, the three analyte test strips 122, 128 include a sample port 130 capable of spreading a blood sample over three separate analyte stacks 132, 133, and 134, while the single analyte dry test strips 124 and 126 contain a sample port, such as 137. The three analyte test strips 122, 128 simultaneously test the sample for the concentration of total cholesterol (TC), the concentration of HDL cholesterol, and the concentration of LDL cholesterol. The foregoing description of the test strips is merely exemplary. Any other dry test strip having any other structure may be used. The test strips according to the invention preferably include labeling, such as 138, which indicates to the analyte which the strip measures.

Tray 102 also comprises scheduling confirmation information 140 which is visible after a strip is removed from the tray. Scheduling confirmation information 140 permits a user to confirm after a strip is removed what strips have been used and the time the strip was scheduled to be used. Scheduling confirmation information 140 preferably includes date confirmation information 142, analyte confirmation information 144, and physician confirmation information 146, which preferably includes the name 147 of the physician that prescribed the test, and physician contact information 148, which may be a phone number, or an address, such as an email address. Scheduling confirmation information 140 is preferably applied to the bottom 149 of the tray well 112 which held the dry test strip. It may be applied, for example, by direct printing on the tray bottom, or printing on a label which is applied to the tray well bottom. Tray 102 preferably is made of plastic, such as polyethylene or EVA but also may be made of foil or other suitable plastic or metal that can be vapor sealed.

The test strips according to the invention also provide analyte indicators, such as 152, 154 and 156 that indicate to the reader the analyte that the strip measures. In the embodiment of FIG. 1, the analyte indicators are protrusions on the end 160 of the test strip which is inserted into the reader. As known in the electronics art, the protrusions can be read optically, can close a circuit in the reader, or utilize some other electronic mechanism to differentiate the strips. The strips may also be identified by a RFID tag 164, a bar code 174 on the strip, or other known way of electrically or optically providing a signal to the electronics of the reader. One skilled in the art will recognize that item 174 is not an actual bar code, as actual bar codes do not meet the requirements for drawings by patent offices, but is intended to represent a bar code. Also, one skilled in the art will recognize that a test strip packaging system 100 may not generally include different identification elements, such as protrusions, a bar code and RFID tag on different strips within a package, but generally each strip within a package will communicate data in the same way. However, different solutions to the communication requirements of the invention are shown in FIG. 1 by way of illustration. It is contemplated by the invention however that multiple communication devices may be included on a single test strip.

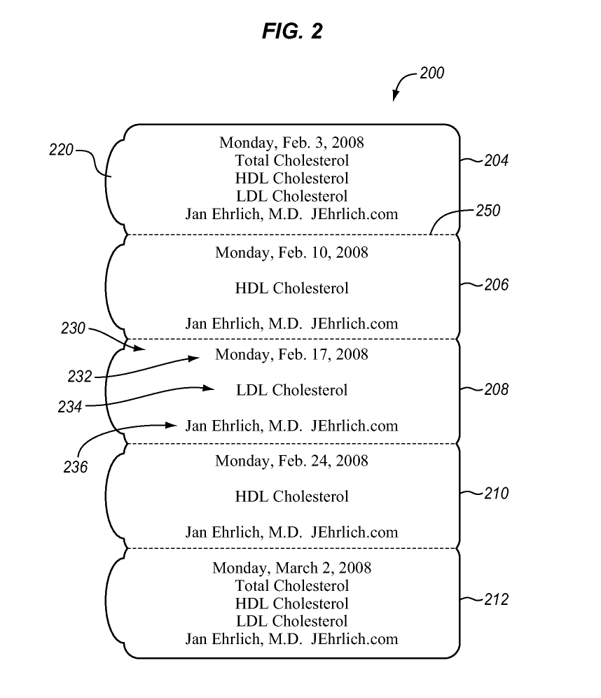

An exemplary completed packaging system 200 according to the invention is shown in FIG. 2. Packaging system or closed container 201 comprises a tray 100 with all five strips in place covered by a cover seal sheet 204. Cover seal sheet is preferably separated into five sections, 204, 206, 208, 210 and 212, by tear lines 250. Each section, such as 204, includes a pull tab 220, which permits the section to be separated from the neighboring section, in this case 206, and removed from tray 102, allowing the strip in the well beneath that section to be accessed. Each section also includes strip-specific scheduling information 230, which preferably matches the scheduling confirmation information on the bottom of the corresponding tray well. Strip-specific scheduling information is contrasted to generalized scheduling information as may be placed on a prescription medicine label. For example, generalized scheduling information on a prescription medicine label may state that one tablet is to be taken each day before bedtime, or a tablet is to be taken twice a day with food. Strip-specific scheduling information provides different information for individual strips within the container. For example, strip-specific scheduling information may provide the date on which each strip is to be used, a date range for use of each strip, or the order in which the strips are to be used. It is noted that while it has been known for fifty-years or more to provide scheduling information for pills that must be taken according to a regular schedule, such as birth control pills, there has been no suggestion in the dry test strip art that strip-specific scheduling information would be useful. Strip-specific scheduling information 230 preferably includes date information 232, analyte information 234, and physician information 236, which preferably includes the name 237 of the physician that prescribed the test, and physician contact information 238, which may be a phone number, or an address, such as an email address. Strip-specific scheduling information 230 is applied to cover seal sheet, preferably printed directly on the sheet, but may also be applied to a label which is applied to the sheet. Or the packaging system 200 may be a blister pack, and the strip-specific scheduling information is printed on a card inserted into the pack. Cover seal sheet 204 is preferably made of polyethylene but also be made of foil or some other suitable plastic or metal which can be sealed vapor tight to tray 102. Preferably, cover seal sheet 204 is sonic welded to tray 102, but also may be glued, or otherwise fastened to the tray. Tear lines are made by partially cutting the line at the same time as printing, or by crimping.

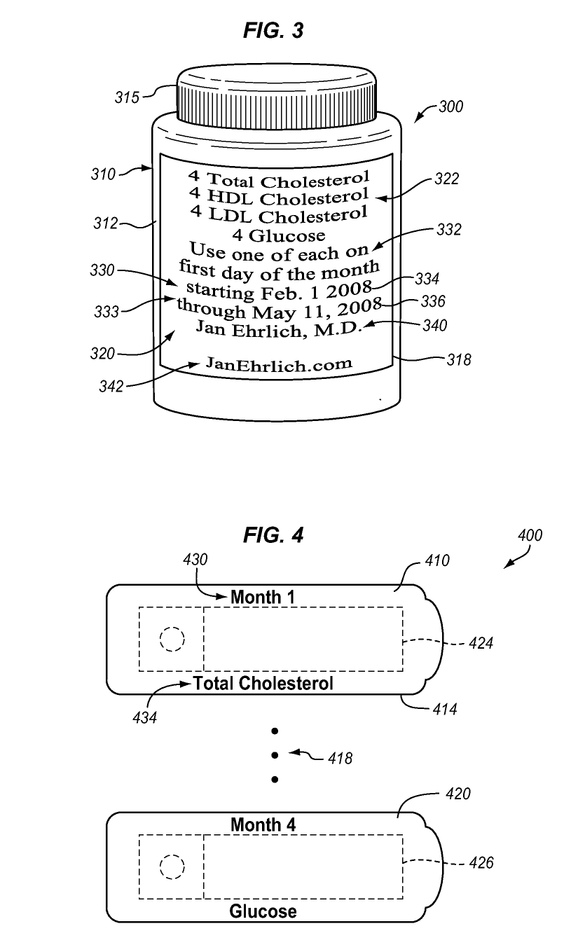

FIG. 3 illustrates another packaging system 300 and method according to the invention. Packaging system 300 comprises sixteen dry test strips, a container 310 comprising a container base 312, a snap-on or screw-on lid 315, and a label 318. User information 320 is applied to the label 318. The dry test strips are not expressly shown in FIG. 3, but are illustrated in FIG. 4. The sixteen dry test strips include four dry test strips of the total cholesterol type, four test strips of the HDL cholesterol type, four test strips of the LDL cholesterol type, and four test strips of the glucose type User information includes contents information 322 and generalized scheduling information 330. Scheduling information 330 preferably includes the directions for use 332 and the schedule period 333, the schedule start date 334, the schedule end date 336, physician information 340 and physician contact information 342.

FIG. 4 illustrates a further dry test strip packaging system 400 according to the invention. The system 400 illustrated is intended to be used in combination with the system 300, though it may be used separately. System 400 includes sixteen dry test strips packets, such as 410 and 420, each separately wrapped in a sealed packet material 414, which packet material may be made of foil, plastic, or other material. Each packet 410, 420 includes a dry test strip 424, 426, respectively, and each packet is labeled with the identification 434 of the analyte and strip-specific scheduling information 430, which, in this embodiment, comprises time indication 430 indicating when each specific strip is to be used. While only two of the packets 410, 420 are shown, the remaining fourteen are indicated by the dots 418.

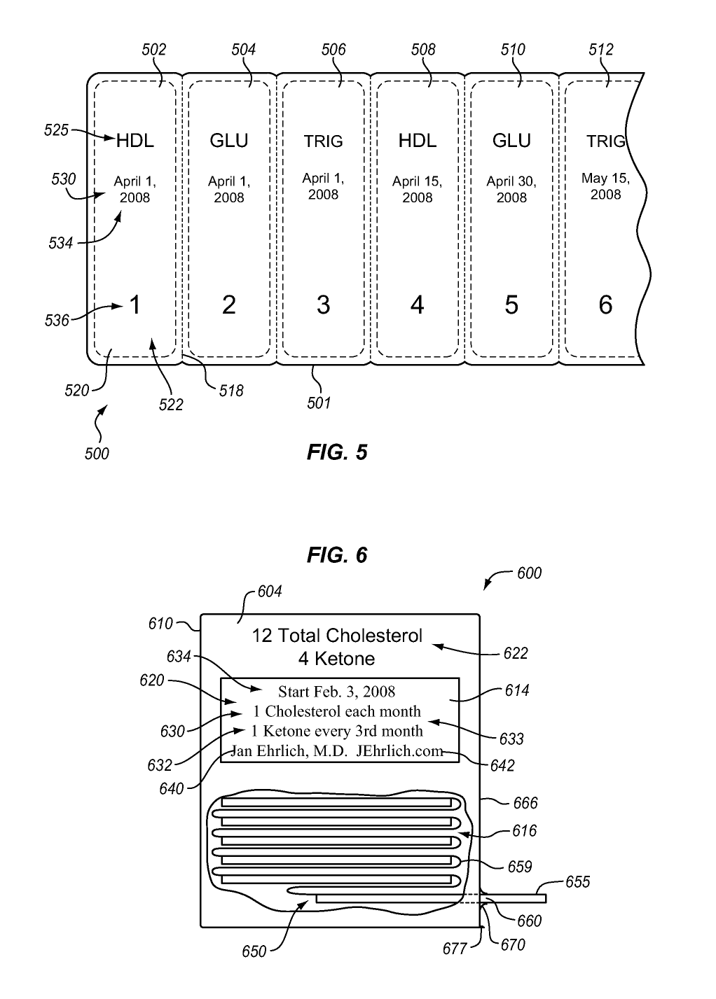

FIG. 5 illustrates another dry test strip packaging system 500 according to the invention that may either be used in combination with the system 300 of FIG. 3 or separately. System 500 comprises a plurality of test strip packets 502, 504, 506, 508, 510, 512, each separately wrapped in a sealed packet material 520, which may be foil, plastic or other sealable material. In embodiment 500 the plurality of packets are joined in a single pack 501, with each packet, such as 502, separable from the others via a tear line, such as 518. Each packet, such as 502, contains labeling information 522, which preferably includes analyte information 525, and scheduling information 530. In system 500, strip-specific scheduling information includes a specific date on which the strip is to be used, 534 and order of use information 536, though only one of the two types of scheduling information may be used. The pack 501 may be rolled up and placed in a container, such as 310, a blister pack, or other suitable container.

Another exemplary embodiment 600 of a dry test strip packaging system according to the invention is shown in FIG. 6. Packaging system 600 comprises a container 610 and a label 614. Container 610 is partially cut away to show a portion of the contents 616. User information 320 is applied to the label 614. User information includes contents information 622 and scheduling information 630. Scheduling information preferably includes the directions for use 632 and the schedule period 633, the schedule start date 634, physician information 640 and physician contact information 642. Contents 616 comprise a dry test strip string 650 including a plurality of dry test strips, such as 655, which are interconnected by connectors, such as 659, which preferably is a ribbon, but may be a string, a tape, or other suitable connecting element. The strips, such as 655, feed through an opening 660, preferably a slot, in the side 666 of container 610. In the preferred embodiment, a resilient slot guard 670 substantially closes around the connector, such as 659, at the end of a strip, which substantially closes the opening 660, and holds the connector. The strip 655 may be disconnected by cutting with a scissors or on a cutting edge 677 attached to container 610. After the connector 659 is cut a small portion of it sticks out of opening 660 and is held in place by opening guard 670. The cut portion sticking out of the guard allows may be grasped and the next strip removed. The strips are serially located in the string 650 in the order in which they are to be used. Each strip includes a labeling 522, such as shown in FIG. 5, with analyte information 525, and strip-specific scheduling information 530. Strip-specific scheduling information 530 preferably includes a date of use, 534 and order of use information 536.

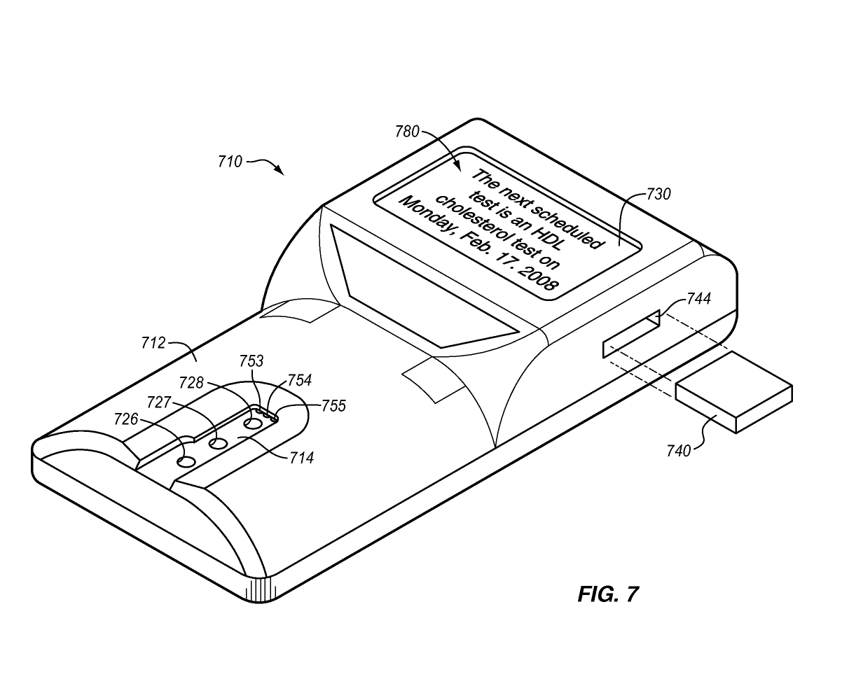

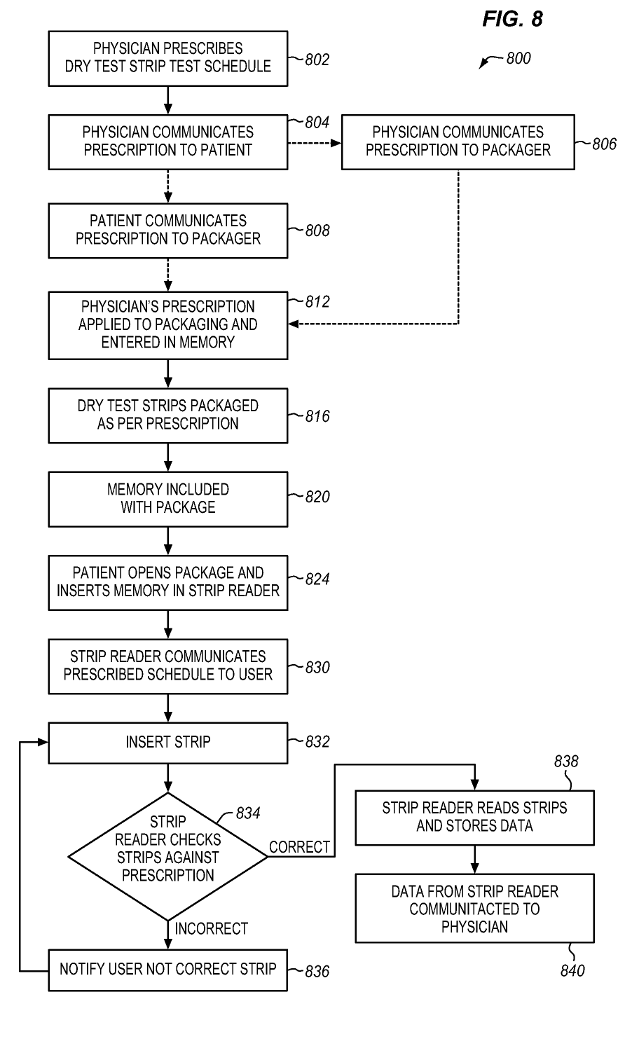

FIG. 7 shows a photometric analyzer 710 according to the preferred embodiment of the invention. The photometric device includes a housing 712 that is sized and configured to be easily hand-held. The housing defines a dry test strip holding region 714 that receives a dry test strip. The holding region 714 includes a number of sensor ports 726, 727 and 728 through which light sources and light sensors integrated into the device 10 interact with the test strip as known in the art. A display 730 provides a visual and/or numeric read-out indicative of the concentration or other parameter of a particular analyte being evaluated. The device 710 includes receptacles 753, 754, and 755 which interact with indicators 156, 154 and 152, respectively, (FIG. 1) to tell the reader what type of test strip is being inserted. The device 10 includes circuitry and a microprocessor configured to analyze the colorimetric response of the reacted test strip according to known techniques. The preferred photometric device 10 and its operation are more fully described in U.S. Pat. No. 5,597,532. However, a computer or other communication device, such as described in U.S. Pat. No. 6,602,469 issued to Christopher T. Maus et al. on Aug. 5, 2003 may also be used. In the preferred embodiment, the device 710 is designed and programmed to operate with the test strips according to the invention. However, any device that has the ability to determine the intensity of light, the frequency or wavelength of light, or other property of light via reflection, scattering or otherwise interaction with a test strip may be used. Other test strips readers and corresponding test strips, for example those that provide an output via a resistance measurement, or any other electrochemical measurement may also be used. See, for example the patents cited in the Background of the Invention. A non-volatile removable storage device 740, preferably a ROM (Read Only Memory) associated with the particular strip to be read is inserted into a memory slot 744 in reader device 10. This storage device 740 contains data relating to the test strip, such as calibration data, as well as data relating to the directions or prescription for using the strip. For example, the storage device 740 may contain information regarding the type of strip, the order in which the strips should be used, and the time of use of each strip. This information can be used to provide communications, such as 780, to the user regarding proper sequencing and timing of the strips. For example, as shown in FIG. 7, if the reader 710 is turned on between scheduled tests, the reader can inform the user of the type of test strip that is to be used next and the time of the next test. In this instance, the reader system 710 informs the user that the next scheduled test is an HDL cholesterol test which is scheduled for Monday, Feb. 17, 2008.

The data mentioned above, such as calibration data, directions or prescription data, data regarding sequencing, or timing, or other information may also be communicated to the reader 710 via other storage devices, such as an RFID tag 164 (FIG. 1), bar code 174, or other optically or electronically readable storage device.

FIG. 8 is a flow chart illustrating an exemplary process 800 for prescribing, packaging and using dry test strips according to the invention. At 802, a physician prescribes one or more dry test strip tests and a schedule for the tests. The prescription is communicated the patient at 804, which may be conventionally via a written prescription, or may be recorded electronically on a card, memory stick, or other electronic storage device. At 806, the physician may also communicate the directions or prescription directly to the packager, which may be a pharmacist, a factory, or a distribution center. One or both of processes 804 and 806 may be used.

If the physician communicates the prescription or other directions to the patient, then the patient may then communicate the prescription to the packager at 808. At 812, the packager applies the prescription of other direction information to the packaging system, such as the system 200, 300, 400, 500, or 600, and/or entered into the memory 740. The dry test strips are then packaged in a container such as 201, 310, 610 as per the prescription or other directions at 816. Preferably, a memory, such as 740, containing the prescription or other direction data is packaged in the container with the strips at 820. The strips are then shipped to the user, or in some cases stored while waiting for sale. It should be understood that in some cases, instead of a prescription or directions from a physician, a standard set of tests following one or more standard test schedules may be packaged, and, in this case, standard directions associated with the strips may be stored on memory 740 and the memory then packaged with the strips as discussed above.

When, at 824, the end user, who may be a patient, obtains the packaged strips, the user opens the container and inserts the memory 740 in the strip reader 710. The reader may download the data in memory 740 to a memory in the reader, or the reader may read the data in the memory only when the data is needed. The strip reader 710 may then communicate, either visually, audibly, or both the test schedule to the user at 830. The user then inserts a strip into the reader at 832. Each time a strip is inserted into strip, the strip reader checks the strips against the prescription or other directions at 834. In the embodiment described, this is done by reading indicators 152, 154 and 156 on the strip. In this embodiment, the indicators are protrusions, each of which activates a switch in reader 710 when they are inserted in receptacles 753, 754 and 755. The indicators may also be mechanical, such as those shown, electrical, such as conductors, optical, such as colored elements or LEDs, or any other suitable indicators. If the strip is not the correct strip, the reader so notifies the user at 836 and the user inserts the correct strip. If the strip is the correct strip, the strip reader reads the strip at 838 and stores the data. Note that, even if the strip is not the correct strip, the reader may note which strip it is, and read the strip and record the data anyway. This is because when a strip is inserted, blood may already have been applied to it and a result for that strip determined, and this prevents waste of a strip. The user may however, insert the strip into the reader prior to use as a check. At 840, the data from the strip is communicated to the user and/or physician. This preferably includes visual communication via the reader display 730, by recording the data in memory 740 and delivering it to the physician's office, by internet, by the user telephoning the physician or by other communication methods.

As discussed above, a feature of the invention is that a plurality of different types of dry test strips are packaged, distributed and sold in a single-user container. “Different types” of dry test strips here means that a first one of said test strips measures a different type of analyte then a second one of said test strips. The limitation of “a single-user container” excludes the situation where a plurality of vials, each containing a number of one type of dry test strip may have been shipped together in another container, since, in that case the plurality of different test strips are in a plurality of containers. Preferably, the single-user container is selected from the group consisting of a bottle, a jar, a vial, a tray, or a blister pack. Preferably, the plurality of dry test strips consists of one-hundred or less of the dry test strips in the single container. More preferably, the plurality of dry test strips consists fifty or less of the dry test strips in the single container. More preferably, the plurality of dry test strips consists twenty-five or less of the dry test strips in the single container. Most preferably, the plurality of dry test strips consists of a dozen or less of the dry test strips in the single container. The forgoing limitations also exclude situations in which a number of different test strips, each type in a different vial, are shipped together, for example in a crate. The container is preferably a container that can be held in one hand by a human being. The container is preferably a single-user container, that is, a container designed to be used by a single user, rather than a container shipped to a distributor, for example. Those skilled in the art understand what is meant by a single-user container.

Another feature of the invention is that a strip-specific schedule for using the plurality of different types of dry test strips is included in the system, the schedule being either in or on the container. A related feature is that the system includes an electronic memory, and the schedule may be stored on the electronic memory. Preferably, the plurality of dry test strips consists of one-hundred or less of the dry test strips in the single container. More preferably, the plurality of dry test strips consists fifty or less of the dry test strips in the single container. More preferably, the plurality of dry test strips consists twenty-five or less of the dry test strips in the single container. Most preferably, the plurality of dry test strips consists of a dozen or less of the dry test strips in the single container.

In another aspect, the invention provides a method of providing scheduling information to a user of a dry test strip, the method comprising storing the scheduling information in an electronic memory; placing the electronic memory in a container with one or more dry test strips; removing the electronic memory from the container; inserting the memory into a dry test strip reader; and communicating the scheduling information to the reader via the dry test strip reader. Preferably, the communicating is selected from the group consisting of audio communication and visual communication. Preferably, the method further comprises passing the scheduling information from a physician to a dry test strip packager, and the placing is performed by the packager. Preferably, the scheduling information is strip-specific scheduling information.

A further feature of the invention is that information concerning the order or use of the test strips and the timing of the use of the test strips appropriate to a particular end user is included in the package. This individualized scheduling information may be provided by the user's physician, and may be communicated to the user via the packager of the test strips.

Once the invention is understood, many new and helpful packaging systems can be imagined. For example, one system might be labeled a statin diagnostic pack. This pack may contain, for example, at least two of an HDL test strip, an LDL test strip and a total cholesterol test strip. Statin drug suppliers may want to include such a pack with their medications, or even give free samples of said packs away. It is known that only 20% of people for which stain drugs are prescribed actually stay on the drug for a year or more. Such statin diagnostic packs would provide an excellent way to show the statin user that the statin drugs are working, and that they should stay on their medications. Such packs would be potential life savers as many of the people that go off their statin drugs become victims of heart attacks.

There has been described a novel dry test strip packaging system and process for prescribing, packaging and using dry test strips that. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiments described, without departing from the inventive concepts. It is also evident that the methods recited may in many instances be performed in a different order; or equivalent structures and processes may be substituted for the various structures and processes described. Consequently, the invention is to be construed as embracing each and every novel feature and novel combination of features present in and/or possessed by the bodily fluid analysis system herein described.

We claim:

1. A system for measuring blood analytes, said system comprising:
a plurality of dry strips, said test strips including a test strip of a first test strip type for measuring a first analyte type and a second test strip of a second test strip type for measuring a second analyte type different from said first analyte type, said types of test strips selected from the group consisting of: test strips for measuring the concentration of high density lipoprotein (HDL) cholesterol, test strips for measuring the concentration of low density lipoprotein (LDL) cholesterol, test strips for measuring the concentration of lipoprotein cholesterol other than HDL and LDL, test strips for measuring total blood cholesterol, test strips for measuring a triglyceride, test strips for measuring glucose, and test strips for measuring a ketone; and
a single-user container of a size that can be held in one hand by a human being, said single-user container containing said first and second test strips.

2. A system as in claim 1 wherein said single-user container is selected from the group consisting of a bottle, a jar, a vial, a tray, or a blister pack.

3. A system as in claim 1 and further comprising strip-specific scheduling information for using said plurality of different types of dry test strips, said strip-specific scheduling information either in or on said container.

4. A system as in claim 3 and further including an optically or electronically readable data storage element, wherein said strip-specific scheduling information is stored in said data storage element.

5. A system as in claim 4 wherein said data storage element is selected from the group consisting of an electronic read-only memory (ROM), a bar code, and a radio frequency identification device (RFID).

6. A system as in claim 1 and further comprising schedule confirmation information which permits a user to confirm after a strip is removed from said container what strips have been used and the time each strip was scheduled to be used.

7. A system as in claim 1 wherein said plurality of dry test strips consists of twenty-five or less of said dry test strips in said single-user container.

8. A system as in claim 1 wherein one of said plurality of dry test strips consists of a three analyte test strip.

9. A system as in claim 1 wherein said plurality of dry test strips comprises a test strip for measuring glucose and a test strip for measuring a ketone.

10. A system as in claim 1 wherein said plurality of dry test strips comprises a test strip for measuring HDL cholesterol, a test strip for measuring glucose, and a test strip for measuring a triglyceride.

11. A system as in claim 1 wherein said plurality of dry test strips comprises an HDL cholesterol test strip, an LDL cholesterol test strip, and a total cholesterol test strip.

12. A method of packaging a dry test strips, said method comprising:
providing a first type of dry test strip selected from the group consisting of: test strips for measuring the concentration of high density lipoprotein (HDL) cholesterol, test strips for measuring the concentration of low density lipoprotein (LDL) cholesterol, test strips for measuring the concentration of lipoprotein cholesterol other than HDL and LDL, test strips for measuring total blood cholesterol, and test strips for measuring a ketone;
providing a second type of dry test strip selected from the group consisting of: test strips for measuring the concentration of high density lipoprotein (HDL) cholesterol, test strips for measuring the concentration of low density lipoprotein (LDL) cholesterol, test strips for measuring the concentration of lipoprotein cholesterol other than HDL and LDL, test strips for measuring total blood cholesterol, and test strips for measuring a ketone, said second type of dry test strip being a different type than said second type of dry test strip; and
packaging said first type of test strip together with said second type of test strip in a single-user container of a size that can be held in one hand by a human being.

13. A method as in claim 12 wherein said packaging comprises sealing each of said test strips in an airtight packet and then packing said packets in said container.

14. A method as in claim 12 and further comprising labeling each of said packets with strip-specific scheduling information.

15. A method as in claim 12 and further comprising storing strip-specific scheduling information in a form accessible by a dry test strip reader, inserting one of said dry test strips in said strip reader, checking said inserted dry test strip against said strip-specific scheduling information, and informing the user of said reader of whether the test strip has been inserted according to said scheduling information.
